(19) United States
(12) Patent Application Publication
(10) Pub. No.: US 2007/0167359 A1
(43) Pub. Date: Jul. 19, 2007

(54) Pharmaceutical composition comprising proteins and/or polypeptides and colloidal particles

(76) Inventors: Moshe Baru, Pardes Hana (IL); Lea Carmel-Goren, Ramat Gan (IL)

Correspondence Address: NATH & ASSOCIATES, 112 South West Street, Alexandria, VA, 22314, US

(21) Appl. No.: 10/553,357

(22) PCT Filed: Apr. 15, 2004
(86) PCT No.: PCT/IL04/00327

§ 371 Date: Jul. 13, 2006

Related U.S. Application Data
(60) Provisional application No. 60/462,701, filed on Apr. 15, 2003.

(51) Int. Cl. A61K 38/17; A61K 9/127

(52) U.S. Cl. 514/12; 424/450; 977/907

(57) ABSTRACT

A pharmaceutical composition for parenteral administration comprising a therapeutically effective amount of a protein or polypeptide and colloidal particles. The particles comprise approximately 1-20 mole percent of an amphipathic lipid derivatized with a biocompatible hydrophilic polymer. The protein or polypeptide is selected from the group consisting of (a) proteins or polypeptides capable of externally binding said colloidal particles; (b) proteins or polypeptides capable of binding polymers of the polyalkylether, polylactic and polyglycolic acid families; and (c) proteins or polypeptides that include a consensus sequence of S/T-X/I/V-I/V/Q/S-S/T-X-X-X-E, where X may be any amino acid, and S,T,L,I,V,E and Q have their standard meanings. The protein or polypeptide is not Factor VIII (FVIII) and is not encapsulated in the colloidal particles.

### FIELD OF THE INVENTION

The present invention relates to a pharmaceutical formulation for the increased half-life, efficacy and stability of various proteins and polypeptides.

### BACKGROUND OF THE INVENTION

Hemophilia A is one of the most frequently occurring inherited coagulation disorders. Patients with hemophilia A are prone to frequent hemorrhages as a result of one or more misfunctions of the coagulation system. One of the causes of hemophilia is a shortage of Factor VIII (FVIII) in the blood. This problem can be treated with Factor VIII concentrates. However, in about 15% of the patients the occurrence results of Factor VIII neutralizing antibodies, so-called inhibitors, whereby a therapy with Factor VIII concentrates is hardly possible.

Two basic approaches have been described in the literature to protect FVIII from inactivation by inhibitors.

WO/80/01456 to Hernker discloses a pharmaceutical composition suitable for oral administration comprising FVIII incorporated within liposomes of 0.5-1.0 microns formed from phospholipids. The phospholipids have a net charge, and the FVIII is incorporated between the layers of the liposome. It is claimed that FVIII levels in the plasma remained above about 5% of the normal value for a period of 50 hours.

U.S. Pat. No. 4,348,384 to Horikoshi states that a composition as described in Hernker was prepared, but did not give satisfactory results. Therefore, Horikoshi incorporates a protease inhibitor into the liposome together with FVIII, in order to protect it from proteolysis. 3% of the normal plasma levels of FVIII were obtained over a period of 6 hours.

U.S. Pat. No. 5,013,556 to Woodle discloses a liposome composition for use in delivering various drugs via the bloodstream. The liposome contains between 1-20 mole percent of an amphipathic lipid derivatized with a polyalkylether. Here also, the drug compound is entrapped within the liposome. These liposome compositions are available commercially under the name of Stealth® vesicles (SUV's, small unilamellar vesicles comprised of phospholipid and polyethylene glycol (PEG) covalently bound to phospholipid).

A further problem with this approach is that liposomes having a large diameter have a short half-life. Therefore, the liposomes must be downsized under high pressure, which can affect protein activities as in coagulation factors V and VIII.

In a second approach, Barrowdliffe, T. W., et al. (1983) J. Lab. Clin. Med. 101:34-43 teaches that mixing FVIII with phospholipid extracted from human and/or animal brain imparts significant protection to the FVIII in vitro. In this approach, the phospholipid is bound to the FVIII rather than encapsulating it. Kemball-Cook, G and Barrowcliffe, T. W. (1992) Thromb. Res. 67:57-71, teaches that a negatively-charged phospholipid surface is necessary for FVIII binding. Negatively charged phosphatidyl serine and phosphatidic acid were found to be highly active in binding to FVIII, while phosphatidyl choline was inactive. However, negatively-charged phospholipids are toxic, and those derived from brain tissue may carry pathogenic agents.

EP 689,428 discloses a liposome composition comprising liposomes having an outer surface layer of hydrophilic polymer chains. A polypeptide or polysaccharide effector molecule is covalently attached to the distal ends of the polymer chains by activation of the lipid anchor prior to effector coupling.

### SUMMARY OF THE INVENTION

It is an object of the present invention to provide a pharmaceutical composition comprising a protein or polypeptide for therapeutic treatment.

It is a further object of the invention to provide a protein or polypeptide in a form having an extended half-life in the bloodstream.

It is a still further object of the invention to provide a method for treating patients suffering from blood coagulation disorders, particularly hemophilia.

In one aspect of the invention, there is provided a pharmaceutical composition for parenteral administration comprising a therapeutically effective amount of a protein or polypeptide and colloidal particles, said particles comprising approximately 1-20 mole percent of an amphipathic lipid derivatized with a biocompatible hydrophilic polymer, wherein said protein or polypeptide is selected from the group consisting of: (a) proteins or polypeptides capable of externally binding said colloidal particles; (b) proteins or polypeptides capable of binding polymers of the polyalkylether, polylactic and polyglycolic acid families; and (c) proteins or polypeptides that include a consensus sequence of S/T-X-L/I/V-I/V/Q/S-S/T-X-X-E, where X may be any amino acid, and S, T, L, I, V, E and Q have their standard meanings. The protein or polypeptide is not encapsulated in said colloidal particles.

In a preferred embodiment, the colloidal particles are substantially neutral and the polymer carries substantially no net charge. In the present specification, the terms “substantially neutral” and “substantially no net charge” mean neither positively nor negatively charged. However, a very low measured charge within experimental error of zero is included within the meaning of the above terms.

The present invention is based on the surprising and unexpected finding that liposomes containing amphipathic lipids derivatized with a bio-compatible hydrophilic polymer can be used to bind proteins or polypeptides, enhance their pharmacokinetic parameters (half-life and area under the curve) and protect them from inhibitors in the bloodstream. This provides a significant advantage over the prior art compositions, since the amphipathic lipids used are synthetic and non-toxic, and can therefore be used in vivo for therapeutic treatment. Furthermore, the liposome does not encapsulate the proteins or polypeptides so that smaller sized liposomes can be used which have a longer half-life in vivo, since they are not removed by the reticuloendothelial system (RES) and the activity of the formulated proteins or polypeptides is not impaired (full activity found in vitro and immediately after injection in vivo). As will be described below in greater detail, the proteins or polypeptides interact non-covalently with the polymer chains on the external surface of the liposomes, and no chemical reaction is carried out to activate the polymer chains, unlike the composition disclosed in EP 689,428.

The term “proteins or polypeptides capable of externally binding said colloidal particles” includes proteins such as coagulation factor VIIa (FVIIa), factor V (FV), factor IX (FIX) and factor X (FX), Granulocyte colony-stimulating factor (G-CSF), Granulocyte macrophage colony-stimulating factor (GM-CSF), Interferon γ and Glucagon-Like Peptide 1 (GLP-1), Interferon-γ and Glucagon-Like Peptide 1 (GLP-1) and a copolymer (Copaxone, Teva, Israel) composed of repeats of 4 amino acids (L-ala, L-glu, L-lys and L-tyr). The identity of these proteins may be ascertained empirically as is known by the skilled man of the art.

The pharmaceutical composition of the invention may be used to treat various diseases, as is known to the skilled man of the art. For example, a composition comprising Copaxone may be used to protect central nervous system (CNS) cells from glutamate toxicity and to treat injury or disease caused or exacerbated by glutamate toxicity. The composition may also be used to treat multiple sclerosis, diabetic neuropathy, senile dementia, Alzheimer's disease, Parkinson's Disease, facial nerve (Bell's) palsy, glaucoma, Huntington's chorea, amyotrophic lateral sclerosis, status epilepticus, non-arteritic optic neuropathy, or vitamin deficiency, as described in US Patent Application No. 20020037848.

The term “proteins or polypeptides capable of binding polymers of the polyalkylether, polylactic and polyglycolic acid families” includes proteins and polypeptides which bind to polymers of the polyalkylether, polylactic and polyglycolic acid families or derivatives thereof by any non-covalent mechanism, such as ionic interactions, hydrophobic interactions, hydrogen bonds and Van der Waals attractions (Arakawa, T. and Timasheff, S. N. (1985) Biochemistry 24:6756-6762; Lee, J. C. and Lee, L. L. Y (1981) J. Biol. Chem. 226:625-631). A preferred example of such a polymer is polyethylene glycol (PEG).

The term “therapeutically effective amount” is to be understood as referring to an amount of protein or polypeptide which results in a level of the protein or polypeptide in the bloodstream having a desired therapeutic effect. Such an amount can be experimentally determined by administering compositions comprising different amounts of the protein or polypeptide and measuring the level in the blood at various times after administration.

The amphipathic lipid used to prepare the colloidal particles may be obtained from either natural or synthetic sources. Most preferred lipids include phospholipids such as phosphatidylcholine and phosphatidylethanolamine, and carbamate-linked uncharged lipopolymers such as aminopropanediol distearoyl (DS) The purpose of the biocompatible hydrophilic polymer is to sterically stabilize the SUVs, thus preventing fusion of the vesicles in vitro, and allowing the vesicles to escape adsorption by the RES in vivo. The polymer will preferably have a molecular weight of between about 500 to about 5000 daltons, most preferably approximately 2000 daltons.

The colloidal particles will preferably have a mean particle diameter of between about 0.03 to about 0.4 microns, most preferably about 0.1 microns. This is to increase their circulation time in vivo and prevent their adsorption by the RES. The amphipathic lipid comprises approximately 0.5 to about 20 mole % of the particles, preferably approximately 1-6%, most preferably 3%.

A variety of known coupling reactions may be used for preparing vesicle forming lipids derivatized with hydrophilic polymers. For example, a polymer (such as PEG) may be derivatized to a lipid such as phosphatidylethanolamine (PE) through a cyanuric chloride group. Alternatively, a capped PEG may be activated with a carbonyl diimidazole coupling reagent, to form an activated imidazole compound. A carbamate-linked compound may be prepared by reacting the terminal hydroxyl of MPEG (methoxyPEG) with p-nitrophenyl chloroformate to yield a p-nitrophenyl carbonate. This product is then reacted with 1-amino-2,3-propanediol to yield the intermediate carbamate. The hydroxyl groups of the diol are acylated to yield the final product. A similar synthesis, using glycerol in place of 1-amino-2,3-propanediol, can be used to produce a carbonate-linked product, as described in WO 01/05873. Other reactions are well known and are described, e.g. in the aforementioned U.S. Pat. No. 5,013,556, whose contents are incorporated herein by reference.

The composition of the invention may be administered by injection, preferably iv, sc or im. The prior art compositions were intended for oral use only, due to side effects caused during injection by the liposome composition. The composition of the invention, on the other hand, is not toxic by injection, apparently due to the small size and lack of toxic phospholipids. Toxicology studies of PEGylated liposomes and FVIII in rats have been carried out—no toxicity was found at doses of 500 units/kg. Amounts of up to 0.5 gm/Kg body weight of colloidal particles according to the invention have been injected without detectable toxic symptoms. Although the free form of the proteins and polypeptides used in the invention have a short half-life in the vascular system, administering them in the composition of the invention is expected to increase their half-life by at least 1.5 times. The composition of the invention is expected to be effective in “on demand” and prophylactic treatment of patients.

In another aspect of the invention, it has been found that there is a consensus sequence of 8 amino acids located within the proteins that binds the liposomes of the invention but is absent in proteins that do not bind the liposomes. This consensus sequence is S/T-X-L/I/V-I/V/Q/S-S/T-X-X-E, where X may be any amino acid, and S, T, L, I, V, E and Q have their standard meanings.

In another aspect of the invention, it has been found that injection of PEGylated liposomes separately from the injection of the protein increases the protein half-life and long-term efficacy.

In a further aspect of the invention, cholesterol is supplemented to the pharmaceutical composition.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 illustrates a real time interaction of PEGylated liposomes to immobilized FVIII. a, PEGylated liposomes, but not control POPC liposomes (PC) and POPC:DSPE liposomes (PC:PE), bind to immobilized rFVIII. b, PEGylated liposomes bind to immobilized rFVIIE (FVIII) but not to immobilized HSA. c, PEGylated liposomes bind to immobilized rFVIII in the absence or presence (Inhibitors) of a diluted (×100) serum of a hemophilia A patient that developed anti FVIII antibodies (titer of 20 Bethesda units/ml). Response is indicated in Resonance Units EU). Response in a-c is corrected for nonspecific binding to HSA coated channel, which was less than 5% Real time interaction of PEGylated liposomes to immobilized FVIII coated channels.

FIG. 2. Real time interaction of PEGylated liposomes to immobilized FIX. a, PEGylated liposomes bind to immobilized FIX but not to immobilized HSA. b, PEGylated liposomes, but not control POPC liposomes, bind to immobilized FIX. Response is indicated in Resonance Units (RU). Response is corrected for nonspecific binding to HSA coated channel, which was less than 5% Real time interaction of PEGylated liposomes to immobilized FIX elative to the binding to FIX coated channels.

FIG. 3. Real time interaction of PEGylated liposomes to immobilized G-CSF. a, PEGylated liposomes bind to immobilized G-CSF but not to immobilized HSA. b, PEGylated liposomes, but not control POPC liposomes, bind to immobilized G-CSF. Response is indicated in Resonance Units (U). Response is corrected for nonspecific binding to HSA coated channel, which was less than 5% Real time interaction of PEGylated liposomes to immobilized G-CSF elative to the binding to G-CSF coated channels.

FIG. 4. Real time interaction of PEGylated liposomes to immobilized GM-CSF. a, PEGylated liposomes bind to immobilized GM-CSF but not to immobilized HSA. b, PEGylated liposomes, but not control POPC liposomes bind to immobilized GM-CSF. Response is indicated in Resonance Units EU). Response is corrected for nonspecific binding to HSA coated channel, which was less than 5% Real time interaction of PEGylated liposomes to immobilized GM-CSF elative to the binding to G-CSF coated channels.

FIG. 5. Real time interaction of PEGylated liposomes to immobilized Interferon γ. a, PEGylated liposomes bind to immobilized INF-γbut not to immobilized HSA. b, PEGylated liposomes, but not control POPC liposomes bind to immobilized INF-γ. Response is indicated in Resonance Units EU). Response is corrected for nonspecific binding to HSA coated channel, which was less than 5% Real time interaction of PEGylated liposomes to immobilized INF-γ elative to the binding to INF-γ coated channels.

FIG. 6. Real time interaction of PEGylated liposomes to immobilized GLP-1. a, PEGylated liposomes bind to immobilized GLP-1 but not to immobilized HSA. b, PEGylated liposomes, but not control POPC liposomes, bind to immobilized GLP-1. Response is indicated in Resonance Units (RU). Response is corrected for nonspecific binding to HSA coated channel, which was less than 5% Real time interaction of PEGylated liposomes to immobilized GLP-1 elative to the binding to GLP-1 coated channels.

FIG. 7. PEGylated liposomes bind to immobilized FIX in the absence or presence Antibodies) of a diluted (×10) rabbit polyclonal anti human FIX antibodies (Sigma).

FIG. 8. Consensus sequence and FVIII binding sites for PEGylated liposomes. a, Consensus sequence for binding PEGylated liposomes in various proteins. Conserved amino acids are depicted in red whereas unconserved amino acids are depicted in blue. b, Human FVIII domain structure. Discrete domain structure of human FVIII as deduced from its primary structure. Liposome binding sites are represented as black squares. Red arrows indicated with T represent thrombin activation sites. c, Binding of PEGylated liposomes or POPC liposomes (PC) to a peptide derived from FVIII sequence (1783-1796 amino acids) immobilized at a CM5 sensor chip. Response is indicated in Resonance Units (RU).

FIG. 9. Real time interaction of PEGylated liposomes to immobilized Copaxone. a, PEGylated liposomes bind to immobilized Copaxone but not to immobilized HSA. b, PEGylated liposomes, but not control POPC liposomes, bind to immobilized Copaxone. Response is indicated in Resonance Units (RU). Response is corrected for nonspecific binding to HSA coated channel, which was less than 5% Real time interaction of PEGylated liposomes to immobilized Copaxone elative to the binding to Copaxone coated channels.

FIG. 10. Survival of hemophilic mice following tail cuts. Kaplan-Meierb plots are shown for hemophilic mice injected with FVIII (Kogenate-FS, Bayer) or FVIII and PEGylated liposomes (injected one hour later). T test statistical analysis indicates statistically significant differences between the groups (P<0.05).

FIG. 11. Real time interaction of PEGylated liposomes composed of POPC:DSPE-PEG 2000 (lipids from Genzyme Pharmaceuticals, Liestal, Switzerland) with 97:3 mole ratio, respectively, and PEGylated liposomes composed of POPC:DS-c-PEG2000 (lipids from Genzyme Pharmaceuticals, Liestal, Switzerland) with 97:3 mole ratio, respectively, to immobilized FVIII.

### DETAILED DESCRIPTION OF THE INVENTION

### 1. Examples 1-8

1.1 Material &Methods

Liposome preparation. Liposomes composed of palmitoyl-oleoyl phosphatidyl-choline (POPC) and distearoyl phosphatidyl-ethanolamine methyl polyethylene glycol 2000 (DSPE-PEG 2000) (Genzyme Pharmaceuticals, Liesatal, Switzerland) (97:3 molar ratio, respectively), POPC and distearoyl phosphatidyl-ethanolamine (DSPE) (Aventi Polar Lipids, Alabaster Ala., USA) (97:3 molar ratio, respectively) were prepared as follows: lipids were dissolved to 10% w/v in tert-buthanol (Reidel-de Haen, Seelze, Germany), frozen and the solution was lyophilized. The resulting dry lipid powder was re-suspended to 2-13% (w/v) in a 50 mM sodium citrate buffer, pH 7.0 to form liposomes. The liposomes were filtered using LiposoFast™-100 extruder apparatus (Aventin Inc., Ottawa, Canada) through polycarbonate filters 1.2 μm, 0.2 μm, 0.1 μm and 0.05 μm in size (Poetics Corp., Livermore Calif., USA) to form liposomes in the size of 80-110 nm. The liposome solution was then passed through a 0.2 μm cellulose acetate sterilizing filter (Sterivex™, Millipore Corporation, Bedford Mass., USA) and stored at 2-8° C.

Real time interactions—Surface Plasmon Resonance analysis. Binding studies were performed using a Biacore™ 2000 biosensor instrument (Biacore AB, Uppsala, Sweden). The following proteins were immobilized onto a CM5-sensor chip (Biacore AB, Uppsala, Sweden) at 1500RU(˜1.5 ng/mm²), by the amine coupling kit as prescribed by the supplier: FVIII (Kogenate-FS, Bayer, Berkley Calif., USA), FIX (Benefix, Genetics Institute, Cambridge Mass., USA), G-CSF, GM-CSF, IFN-γ, Erytiropoietin, Human Growth Hormone, Interferon-alpha 2a, Interferon-alpha 2b (ProSpec-Tany TechnoGene Ltd, Nes Ziona, Israel), GLP-1, Insulin (Sigma), Copaxone (Teva Pharm, Israel), IgG and HSA (Omrix, Tel-Aviv, Israel). SPR analysis was assessed in 50 mM Na-citrate buffer (pH 7.0) at 25° C. with a flow rate of 10 μl/min for 4 min using either PEGylated-liposomes or control-liposomes in a final concentration of 0.2-2 mM. Regeneration of the sensor chip surface was performed by washing the chip with 1 mM NaOH for 1 min at a flow rate of 10 μl/min. Analysis of SPR data for association, dissociation and affinity constants was carried out by BIA evaluation software (Biacore AB, Uppsala, Sweden).

Multiple Alignments—Multiple sequence alignment was carried out using MUSCA software (http://cbcsrv.watson.ibm.com). The following Swiss-Prot data base accession numbers were used for the multiple alignments: Human (h) FVIII P00451), hFIX (P00740), hG-CSF (P09919), hGM-CSF (P04141), hIFN-γ (P101579), and hGLP-1 (P001275).

1.2 Results:

Binding of Proteins/Peptides to PEGylated Liposomes

We analyzed the binding of proteins and peptides to PEGylated liposomes by Surface Plasmon Resonance (SPR) measurement using a Biacore instrument (Biacoe, Uppsala, Sweden). We immobilized proteins/peptides on a CM5 sensor chip (Biacoe, Uppsala, Sweden), then injected PEGylated liposomes or control liposomes of the same size (80-110 nm) and concentration and measured and analyzed the binding of protein/peptide to the flowed intake liposomes.

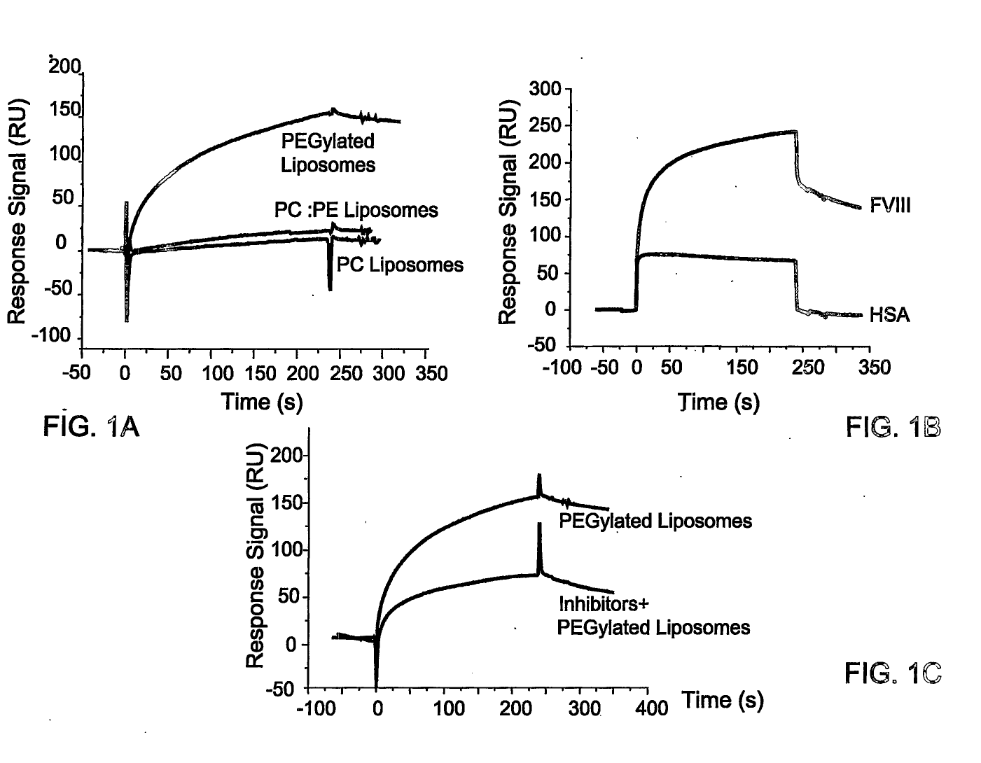

PEGylated liposomes composed of POPC and DSPE-PEG2000 bind to FVIII (FIG. 1a). The binding was dependant on the PEG polymer attached to DSPE lipid since two types of control liposomes composed of POPC and POPC:DSPE did not bind to FVIII (FIG. 1a). In addition, the binding was specific to FVIII, since the PEGylated liposomes did not bind to human serum albumin (HSA) (FIG. 1b). Binding analysis of a representative curve (FIG. 1a) using a two-site binding model indicates that the PEGylated liposomes bind to two sites on FVIII with association rate constants (Kₒₙ) of 3.83×10⁵ and 3.37×10⁶ M−1 s−1, dissociation rate constants (Koff) of 1.72×10⁻³ and 6.6×10⁻³ s−1 and affinity constant (Kd) values of 1.96 nM and 4.5 nM respectively (Table 1).

Polyclonal anti human factor VIII antibodies [serum of a patient that developed anti FVIII antibodies (inhibitors)] compete with the binding of the PEGylated liposomes to both sites (FIG. 1c). Control experiment indicates that total human immunoglobulin G (IgG) did not compete with the binding of FVIII to PEGylated liposomes (data not shown). This indicates that the PEGylated liposomes specifically bind to the same protein domains as anti human factor VIII antibodies.

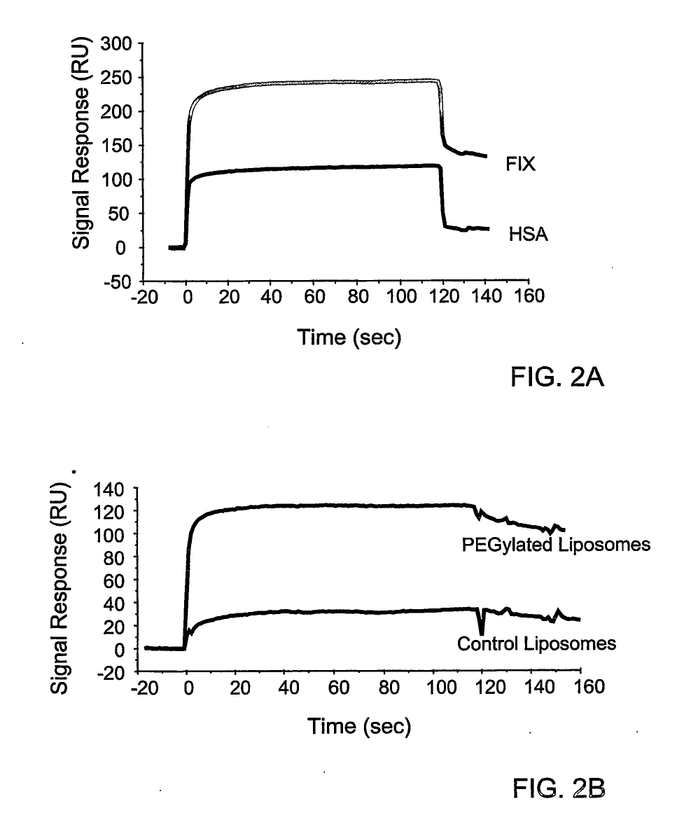

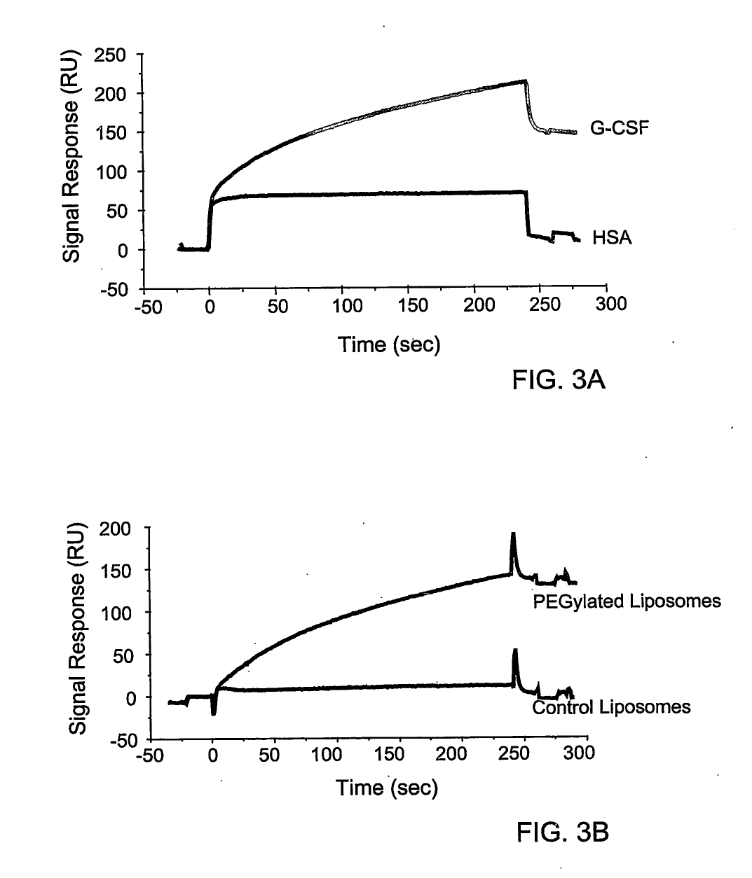

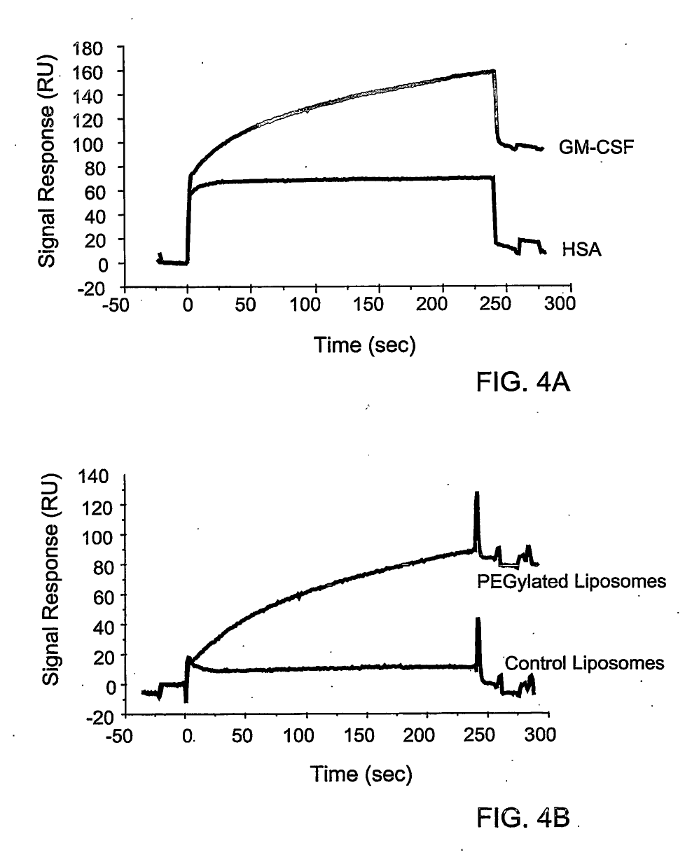

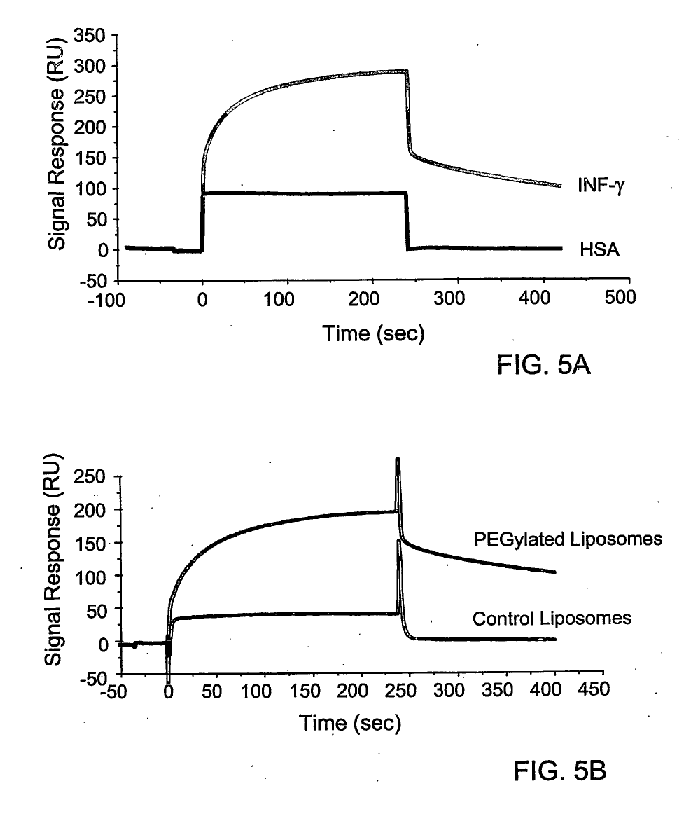

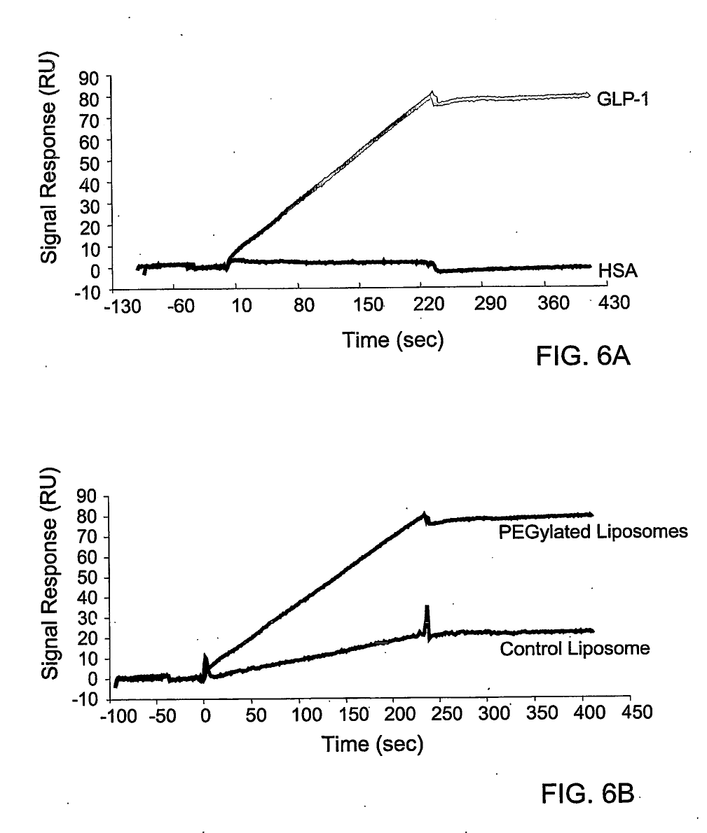

SPR measurements were performed with several additional recombinant and purified proteins. The following proteins were found bind to PEGylated liposomes: Factor IX (FIX), Granulocyte colony-stimulating factor (G-CSF), Granulocyte macrophage colony-stimulating factor (GM-CSF), Interferon γ and Glucagon-Like Peptide 1 (GLP-1) (FIG. 2-6). In addition, we found that the following proteins do not bind to the PEGylated liposomes: HSA, IgG, Insulin, Interferon α 2a, Interferon α 2b, Human growth hormone and Erythropoietin.

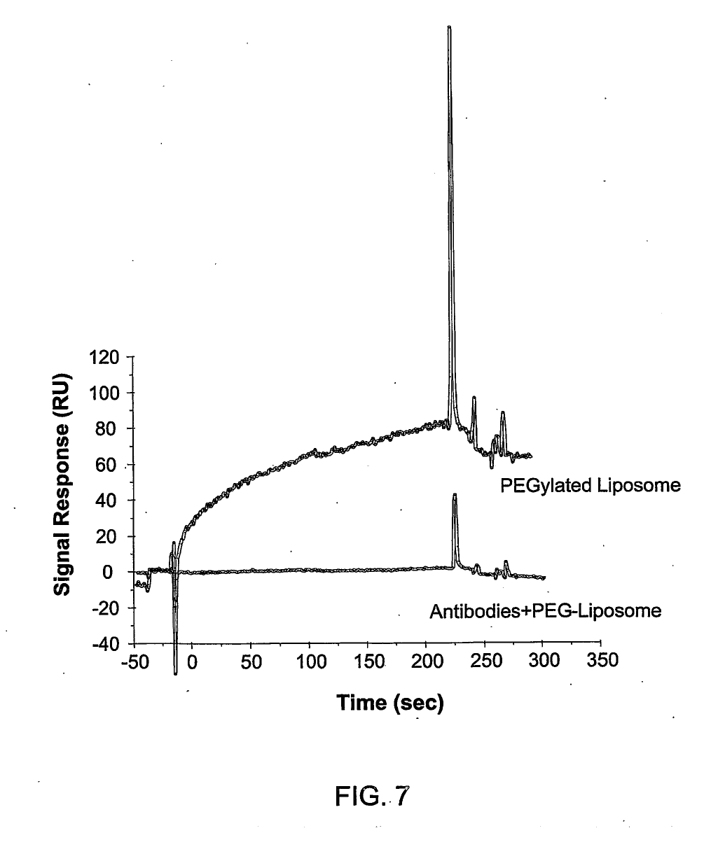

In addition, polyclonal anti human factor IX antibodies (Sigma) compete with the binding of the PEGylated liposomes to both sites (FIG. 7). This indicates that the PEGylated liposomes specifically bind to the same protein domains as anti human factor IX antibodies.

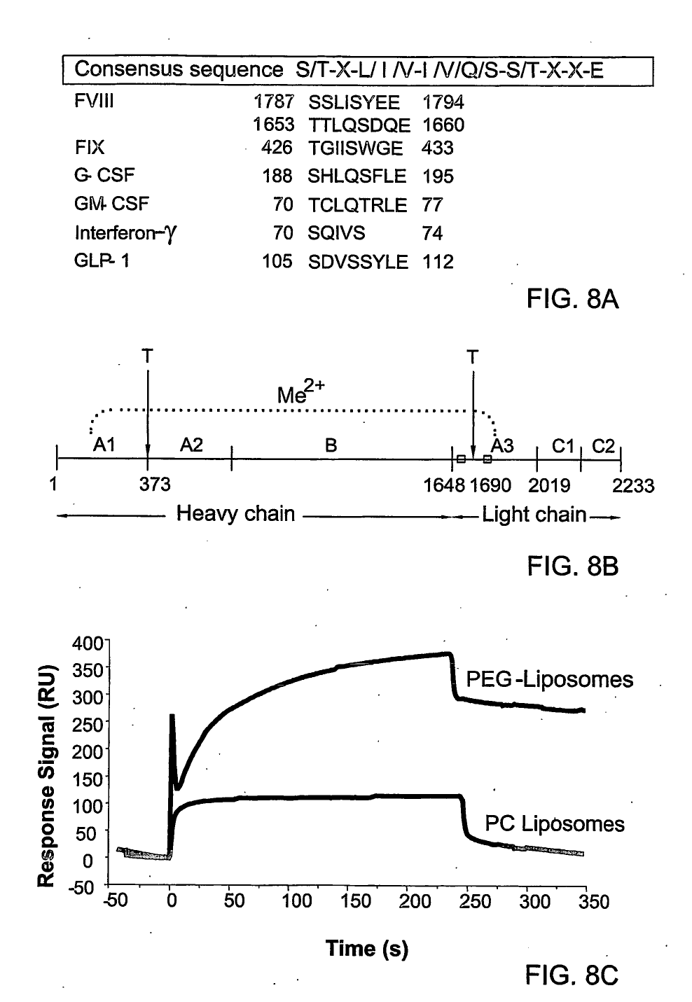

Amino acid sequence analysis indicate that there is a consensus sequence of 8 amino acids (S/T-X-L/I/V-I/V/Q/S-S/T-X-X-E) located within the proteins that binds the PEGylated liposomes (FIG. 8a) but is absent in proteins that do not bind the PEGylated liposomes. To test the consensus sequence and the identified binding sites on FVIII, we synthesized a peptide derived from amino acids 1783-1796 of FVIII and measured its binding to PEGylated liposomes. The peptide binds to PEGylated liposomes, but not to control POPC liposomes, with a Kd of 2.25 nM (FIG. 8c) which is similar to the Kd values of FVIII and PEGylated liposomes that were previously found (Table 1).

A summary of Kₒₙ K_{off} and Kd values of the various proteins and peptide is shown in Table 1.

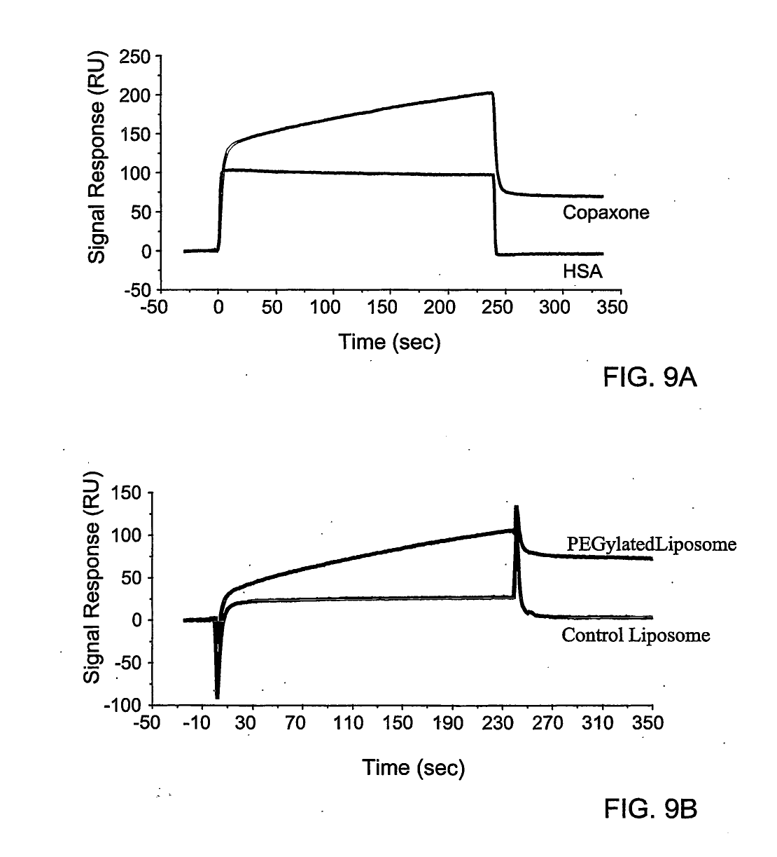

However, Copaxone (Teva, Israel), a synthetic random copolymer composed of repeats of 4 amino acids (L-ala, L-glu, L-lys and L-tyr) but does not contain the consensus sequence, also binds PEGylated liposomes (FIG. 9)

TABLE 1
Kinetic parameter for the binding of proteins/peptide
to PEGylated liposomes.

| Protein/peptide | | kₒₙ (S⁻¹) | k_{off} (M⁻¹S⁻¹) | K_{d} (M) |
|---|---|---|---|---|
| Factor VIII | (1) | 3.83 × 10⁵, | 1.72 × 10⁻³, | 1.96 * 10⁻⁹, |
| | (2) | 3.37 × 10⁶ | 6.6 × 10⁻³ | 4.5 * 10⁻⁹ |

| | | | |
|---|---|---|---|
| Factor IX | 3.33 * 10⁶ | 0.021 | 6.3 * 10⁻⁹ |
| G-CSF | 4.04 * 10⁵ | 0.0137 | 3.39 * 10⁻⁸ |
| GM-CSF | 8.5 * 10⁵ | 0.0103 | 1.21 * 10⁻⁸ |
| INF-γ | 1.44 * 10⁶ | 4.16 * 10⁻³ | 2.88 * 10⁻⁹ |
| GLP-1 | 1.16 * 10⁵ | 2.21 * 10⁻⁴ | 1.91 * 10⁻⁹ |

Association and dissociation of various proteins and a peptide to PEGliposomes were assessed, as described in “material and methods”. The PEGliposome concentrations tested were 18.355 nM for FVIII, FIX, G-CSF, GM-CSF, INF-γ and 183.55 pM for GLP-1. The data obtained for all the proteins were analyzed to calculate association rate constants (kₒₙ), dissociation rate constants (k_{off}) and affinity constants (K_{d}) by BIAevaluation software.

### 2. Examples 9-10

2.1 Material & Methods

Formulation of FIX and G-CSF with PEGylated liposomes. PEGylated liposomes were formulated with either FIX (Octanine, Octapharma) or G-CSF (ProSpec-Tany TechnoGene Ltd, Nes Ziona, Israel) by dissolving the protein with liposome solution (one ml liposome solution/200 units of FIX and 1 ml of liposome solution/10 μg of G-CSF). The vial was incubated on a SRT1 roller mixer rotate at 33 rpm, amplitude 16 mm (Stuart Scientific, Rehill, UK) for 10 minutes (G-CSF) or 60 minutes (FIX), at room temperature (20-25° C.).

Pharmacokinetics of liposome-formulated G-CSF and free G-CSF in mice. Two groups of C57 black mice were subcutaneously (s.c.) injected with: a) 50 μl of G-CSF formulated with PEGylated liposomes (10 μg/ml). b) 50 μl of G-CSF dissolved in 50 mM Na-citrate buffer (10 μg/ml). Mice were bled from retro-orbital sinus into microcentrifuge tubes containing sodium citrate (20 mM final concentration) at various times post injection and plasma was separated by centrifigation at 2,700×g for 10 minutes at 4° C. G-CSF concentrations in mouse plasma was measured by ELISA (G-CSF DouSet ELISA kit, R&D) according to manufacturer instruction

Pharmacokinetics of PegLip-FIX and free FIX in hemophilic mice. Three groups of C57BL mice were injected IV into the tail vein with 50 μl of: a) 200 units/ml PegLip-FIX. b) un-formulated FIX. c) buffer. Mice were bled from retro-orbital sinus into microcentrige tubes containing sodium citrate (20 mM final concentration) at various times post injection and plasma was separated by centrifugation at 2,700×g for 10 minutes at 4° C. FIX activity in mouse plasma was measured by one-stage clotting assay (using Stago reagents and ST4 clotting machine) according to manufacturer's instruction. Since C57BL mice have endogenous FIX activity in their plasma, this endogenous activity (as measured in the control mice) was subtracted from the activity measured at each time-point in the treated groups.

Pharmacokinetics Analysis

Pharmacokinetic parameters (half life and AUC) were analyzed by computer software (RSTRIP, MicroMath Inc.).

Statistic Analysis

Student's t-test.

2.2 Results

Pharmacokinetics of Liposome-Formulated Proteins in Mice

Pharmacokinetic parameters of free and liposome formulated FIX and G-CSF were measured in mice. The results presented in Tables 2-3 indicate that the half-life and area under the curve of the proteins formulated with PEGylated liposomes were higher than that of free proteins.

TABLE 2
Pharmacokinetic parameters following SC injection of liposome-
formulated G-CSF (PegLip-G-CSF or free G-CSF into mice

| | T = 1.75 hr | T = 3.75 hr | T = 5.75 hr | T = 7.75 hr | T = 9.33 hr | HL (hr.) | AUC (pg * h/ml) |
|---|---|---|---|---|---|---|---|
| PegLip- | 58150.72 ± | 56023.32 ± | 48901.71 ± | 24318.43 ± | 14006.75 ± | 5.038 | 434386.17 ± |
| G-CSF | 6299.95 | 7372.35 | 8991.09 | 6512.68 | 4278.52 | 0.896 | 55926.46 |

| | T = 2 hr | T = 4 hr | T = 6 hr | T = 8 hr | T = 9.58 hr | | |
|---|---|---|---|---|---|---|---|
| G-CSF | 62419.23 ± | 50556.82 ± | 37141.44 ± | 12705.32 ± | 5735.84 ± | 3.181 ± | 420746.83 ± |
| | 5212.47 | 6928.12 | 6068.82 | 2780.72 | 284.49 | 0.354 | 40361.42 |

G-CSF was reconstituted with 50 mM Na-citrate buffer pH 7 or formulated with PEGylated liposomes and injected (50 μl) s.c. into mice (6 mice in a group). Human G-CSF concentration (pg/ml) in the plasma was measured by an Enzyme Linked Immunosorbent Assay (Elisa). Pharmacokinetic parameters were calculated for each mouse. Values are means±standard deviation. Student t-test analysis for half-life of PegLip-G-CSF versus G-CSF, P<0.002.

TABLE 3
Pharmacokinetic parameters following IV injection of Factor IX or
PEGylated liposome-formulated Factor IX (PegLip-FIX) into mice.

| | | | | | AUC |
|---|---|---|---|---|---|
| | | | | | (pg * |
| | T = 10 min | T = 4.83 hr | T = 19.83 hr | HL (hr.) | h/ml) |
| PegLip- | 3.78 ± 0.51 | 0.77 ± 0.41 | 0.092 ± 0.1 | 11.72 | 2.02 |
| FIX | | | | | |
| | T = 10 min | T = 5 hr | T = 29 hr | | |
| FIX | 3.85 ± 0.39 | 0.63 ± 0.31 | 0.081 ± 0.078 | 10.96 | 1.85 |

Pharmacokinetic parameters were calculated for each mouse. Values are means±standard deviation. Student t-test analysis for half-life of PegLip-FIX versus FIX, P<0.15.

### 3. Example 11

3.1 Pharmacokinetics and Biological Activity of FVIII Formulated In-Vivo with PEGylated Liposomes in Hemophilic Mice

Pharmacokinetics and biological activity of factor VIII, that was formulated in-vivo with PEGylated liposomes by injection of liposomes 1 hour after the injection of unformulated factor VIII, was measured in hemophilic mice. Hemophilic mice were injected with: a) free (unformulated) FVIII. b) Free FVIII and one hour later a second injection of PEGylated liposomes. The mice were bled at various times post-injection FVIII activity was measured by a clotting assay. In order to test in-vivo efficacy of liposome-formulated FVIII in stopping bleeding and compare it to that of free FVIII tails of the injected mice were cut several times post injection and the survival of the injected mice was measured.

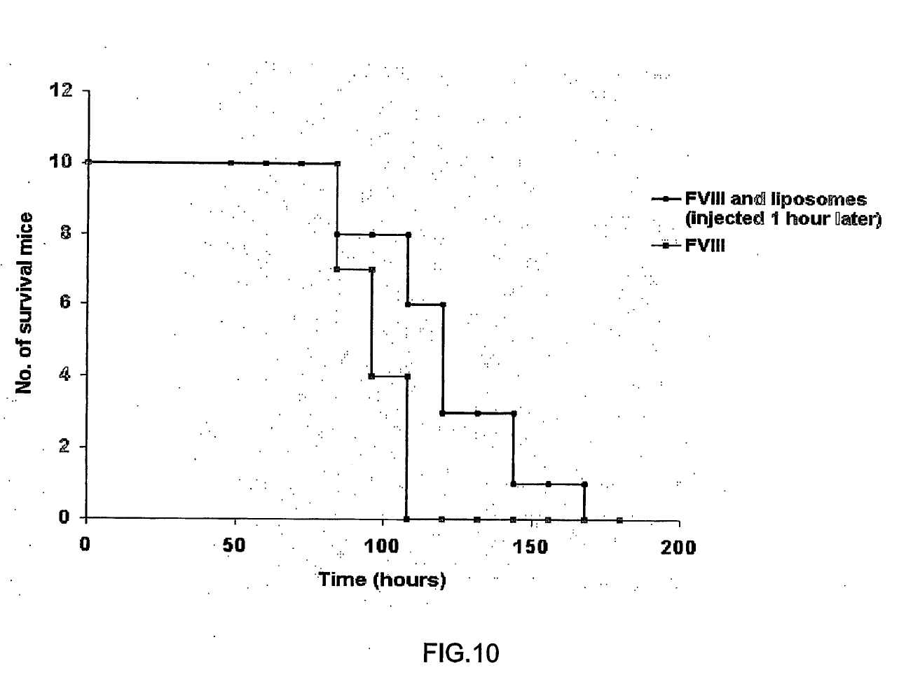

The results presented in Table 4 and FIG. 10 indicate that the half-life and area under the curve of factor VIII that was formulated in-vivo with PEGylated liposomes were higher than that of free FVIII. Accordingly, the survival of mice injected with FVIII and 1 hour later with liposomes were significantly higher.

TABLE 4
Factor VIII activity (IU/ml) and pharmacokinetic parameters following
injection of FVIII or FVIII and 1 hour later PEGylated liposomes into hemophilic mice

| | | | | | | | | Area |
|---|---|---|---|---|---|---|---|---|
| | | | | | | | | under |
| Injection | Time Post | | | | | | Half | the |
| material | injection | T = 10 min | T = 4.6 h | T = 8.3 h | T = 24.6 h | T = 28.8 h | Life | curve |
| FVIII and | Average | 2.03 | 1.35 | 0.975 | 0.66 | 0.61 | 14.5 | 28.8 |
| liposomes | (u/ml) | | | | | | | |
| (Injected 1 | | | | | | | | |
| hour later) | | | | | | | | |
| FVIII | Average | 2.17 | 1.4 | 0.95 | 0.46 | 0.41 | 9.3 | 24.0 |
| | (u/ml) | | | | | | | |

Recombinant FVIII (Kogenate-FS, Bayer) was reconstituted with water and injected (40 μl) into the tail vein of hemophilic mice (10 mice in each group). One our later, PEGylated liposomes (9% lipids, w/v) were injected i.v. (40 μl) into one group of mice. FVIII activity was measured on pool plasma sample of each time-point by a one-stage clotting assay. Pharmacokinetic parameters (half life and AUC) were analyzed by a computer program (RSTRIP, MicroMath Inc.).

### 4. Example 12

The binding of FVIII to liposomes composed of POPC and carbamate-linked uncharged lipopolymer was compared to the binding of FVIII to liposomes composed of POPC and DSPE-PEG by real time interaction analysis. The schematic structure of carbamate-linked uncharged lipopolymer: MPEG aminopropanediol disrearoyl (DS-c-PEG) and 1,2 distearoyl-sn-glycero-3-phosphatidylethanolamine-N-methoxy polyethylene glycol (DSPE-PEG) are shown below.

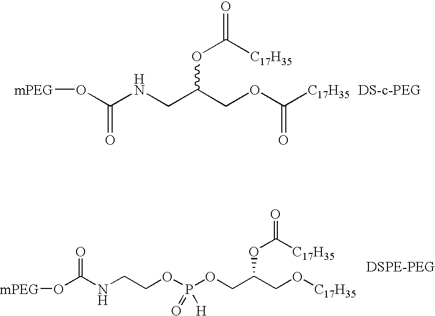

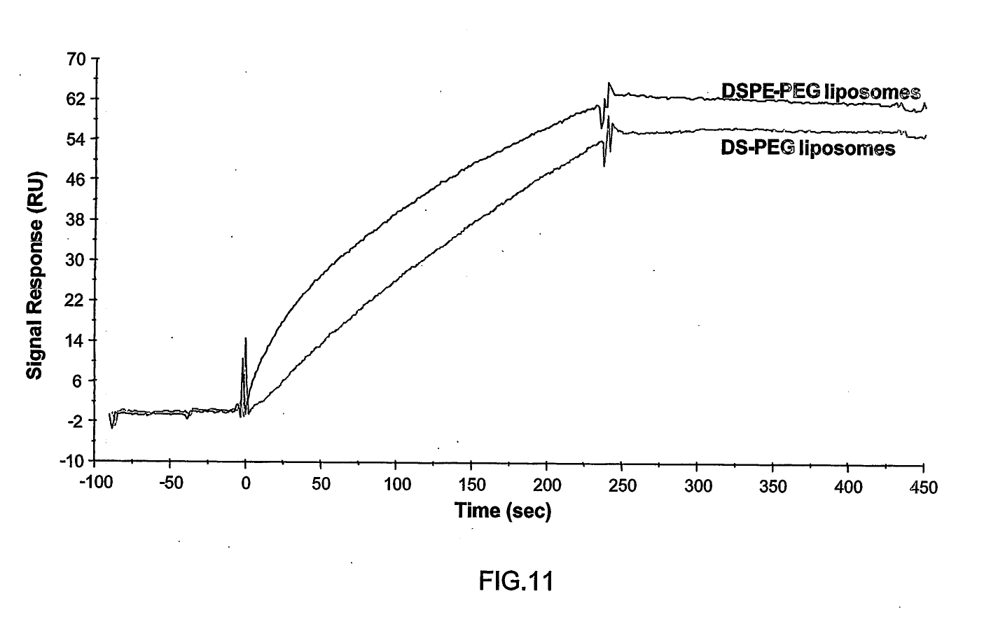

The results are presented in FIG. 11 and show that both types of liposomes interacts with FVIII.

### 5. Example 13

Coagulation factor VIIa is generally used to treat hemophilia patients with inhibitors and to stop trauma bleeding (e.g. war injuries, car accidents). Pharmacokinetics of factor VIIa, formulated with PEGylated liposomes, was measured in rats. Sprague Dawley (SD) rats (180 g) rats were injected with 36 μg/rat of free (unformulated) FVIIa (Nova Nordisk) or FVIIa formulated with PEGylated liposomes. The rats were bled at various times post-injection and FVIIa activity was measured by a clotting assay (Stago). The results presented in Tables 5 and 6 indicate that the half-life and area under the curve of factor VIIa that was formulated in-vivo with PEGylated liposomes were higher than that of free FVIIa.

TABLE 5
Factor VIIa activity (IU/ml) and pharmacokinetics following injection
of FVIIa or FVIIa formulated with pegylated liposomes into rats

| | | | | | | | | Area |
|---|---|---|---|---|---|---|---|---|
| | Time | | | | | | Half | under the |
| Injected | post- | | | | | | Life | curve |
| material | injection | T = 10 min | T = 3 hrs | T = 7 hrs | T = 24 hrs | T = 30 hrs | (HL) (h) | (AUC) |
| PEGLip- | Average | 573 ± 255 | 38 ± 8 | 7 ± 1 | 1.6 ± 1 | 0.5 ± 0.2 | 3.08 | 2013 |
| rFVIIa | (U/ml) ± SD | | | | | | | |
| (n = 4) | | | | | | | | |
| rFVIIA | Average | 706 ± 45 | 45 ± 3 | 4.6 ± 1 | 0.1 ± 0.1 | 0 | 1.86 | 1411 |
| (n = 4) | (U/ml) ± SD | | | | | | | |

TABLE 6
Factor VIIa activity (IU/ml) and pharmacokinetics following injection
of FVIIa or FVIIa formulated with pegylated liposomes into rats
(pooled samples)

| | | | | | | | Area |
|---|---|---|---|---|---|---|---|
| | | | | | | Half | under the |
| Injected | | | | | | Life | Curve |
| material | T˜10 min | T˜3 hrs | T˜7 hrs | T˜23 hrs | T˜31 hrs | (HL) (h) | (AUC) |
| PEGLip- | 287 | 22.6 | 8.7 | 1.6 | 1.2 | 3.84 | 1019 |
| rFVIIa | | | | | | | |
| (n = 3) | | | | | | | |
| rFVIIa | 319 | 27 | 5 | 0 | 0 | 1.15 | 513 |
| (n = 3) | | | | | | | |

1-27. (canceled)

28. A pharmaceutical composition for parenteral administration comprising a therapeutically effective amount of a protein or polypeptide and colloidal particles, said particles comprising approximately 1-20 mole percent of an amphipathic lipid derivatized with a biocompatible hydrophilic polymer,
wherein said protein or polypeptide is selected from the group consisting of:
(a) proteins or polypeptides capable of externally binding said colloidal particles;
(b) proteins or polypeptides capable of binding polymers of the polyalkylether, polylactic and polyglycolic acid families; and
(c) proteins or polypeptides that include a consensus sequence of S/T-X-L/I/V-I/V/Q/S-S/T-X-X-E, where X may be any amino acid, and S, T, L, I, V, E and Q have their standard meanings;
wherein said protein or polypeptide is not Factor VIII (FVIII),
and wherein said protein or polypeptide is not encapsulated in said colloidal particles.

29. The pharmaceutical composition of claim 28 wherein said colloidal particles are substantially neutral and said polymer carries substantially no net charge.

30. The pharmaceutical composition of claim 28 wherein said colloidal particles have a mean particle diameter of between about 0.03 to about 0.4 microns.

31. The pharmaceutical composition of claim 30 wherein said colloidal particles have a mean particle diameter of approximately 0.1 microns.

32. The pharmaceutical composition of claim 28 wherein said amphipathic lipid is a phospholipid from natural or synthetic sources.

33. The pharmaceutical composition of claim 32 wherein said amphipathic lipid is phosphatidylethanolamine (PE).

34. The pharmaceutical composition of claim 28 wherein said amphipathic lipid is a carbamate-linked uncharged lipopolymer.

35. The pharmaceutical composition of claim 34 wherein said amphipathic lipid is aminopropanediol distearoyl (DS).

36. The pharmaceutical composition of claim 28 wherein said colloidal particles further comprise a second amphipathic lipid obtained from either natural or synthetic sources.

37. The pharmaceutical composition of claim 36 wherein said second amphipathic lipid is phosphatidylcholine.

38. The pharmaceutical composition of claim 36 wherein cholesterol is supplemented to the composition.

39. The pharmaceutical composition of claim 28 wherein said biocompatible hydrophilic polymer is selected from the group consisting of polyalkylether, polylactic and polyglycolic acid families.

40. The pharmaceutical composition of claim 39 wherein said biocompatible hydrophilic polymer is polyethylene glycol.

41. The pharmaceutical composition of claim 40 wherein the polyethylene glycol has a molecular weight of between about 500 to about 5000 daltons.

42. The pharmaceutical composition of claim 41 wherein the polyethylene glycol has a molecular weight of approximately 2000 daltons.

43. The pharmaceutical composition of claim 28 wherein said protein or polypeptide is selected from the group consisting of prothrombin, Factor VIIa, Factor X, Factor V, Factor IX (FIX), granulocyte colony-stimulating factor (G-CSF), granulocyte macrophage colony-stimulating factor (GM-CSF), interferon γ, glucagon-like peptide 1 (GLP-1) and Copaxone.

44. The pharmaceutical composition of claim 43 wherein the polypeptide is Copaxone and the composition may be used for the treatment of a disease selected from multiple sclerosis, diabetic neuropathy, senile dementia, Alzheimer's disease, Parkinson's Disease, facial nerve (Bell's) palsy, glaucoma, Huntington's chorea, amyotrophic lateral sclerosis, status epilepticus, non-arteritic optic neuropathy and vitamin deficiency.

45. The pharmaceutical composition of claim 43 wherein the polypeptide is Factor VIIa and the composition may be used in hemophilia patients with inhibitors and for the treatment of trauma bleeding.

46. The pharmaceutical composition of claim 28 wherein the protein or polypeptide comprises the amino acid consensus sequence of S/T-X-L/I/V-I/V/Q/S-S/T-X-X-E, where X is any amino acid, and S, T, L, I, V, E and Q have their standard meanings.

47. Method of treatment of a patient suffering from a disease comprising administrating to said patient a pharmaceutical composition for parenteral administration comprising a therapeutically effective amount of a protein or polypeptide effective in the treatment of the disease and colloidal particles, said colloidal particles comprising approximately 1-20 mole percent of an amphipathic lipid derivatized with a biocompatible hydrophilic polymer,
wherein said protein or polypeptide is selected from the group consisting of:
(a) proteins or polypeptides capable of externally binding said colloidal particles;
(b) proteins or polypeptides capable of binding polymers of the polyalkylether, polylactic and polyglycolic acid families; and
(c) proteins or polypeptides that include a consensus sequence of S/T-X-L/I/V-I/V/Q/S-S/T-X-X-E, where X may be any amino acid, and S, T, L, I, V, E and Q have their standard meanings;
wherein said protein or polypeptide is not Factor VIII (FVIII),
and wherein said protein or polypeptide is not encapsulated in said colloidal particles.

48. The method of claim 47 wherein said disease is hemophilia.

49. The method of claim 47 wherein said patient has developed inhibitor antibodies to said protein or polypeptide.

50. Method of treatment of a patient suffering from a disease comprising administrating to said patient a pharmaceutical composition for parenteral administration comprising a therapeutically effective amount of a protein or polypeptide effective in the treatment of the disease and colloidal particles, said colloidal particles comprising approximately 1-20 mole percent of an amphipathic lipid derivatized with a biocompatible hydrophilic polymer,
wherein said protein or polypeptide is selected from the group consisting of:
(a) proteins or polypeptides capable of externally binding said colloidal particles;
(b) proteins or polypeptides capable of binding polymers of the polyalkylether, polylactic and polyglycolic acid families; and
(c) proteins or polypeptides that include a consensus sequence of S/T-X-L/I/V-I/V/Q/S-S/T-X-X-E, where X may be any amino acid, and S, T, L, I, V, E and Q have their standard meanings;
wherein said colloidal particles and said protein or polypeptide are administered separately,
and wherein said protein or polypeptide is not encapsulated in said colloidal particles.

51. The method of claim 50 wherein said protein or polypeptide is not Factor VIII (FVIII).

52. The method of claim 50 wherein said colloidal particles comprise liposomes and said protein or polypeptide is Factor VIII (FVIII).
